(19) United States
(12) Patent Application Publication
(10) Pub. No.: US 2014/0031341 A1
(43) Pub. Date: Jan. 30, 2014

(54) MODULATORS AND METHODS FOR THE TREATMENT OF ROSACEA

(71) Applicants: Jerome Aubert, Grasse (FR); Martin Steinhoff, San Francisco, CA (US)

(72) Inventors: Jerome Aubert, Grasse (FR); Martin Steinhoff, San Francisco, CA (US)

(73) Assignees: UNIVERSITAT MUNSTER, Munster (DE); GALDERMA RESEARCH & DEVELOPMENT, Biot (FR)

(21) Appl. No.: 13/997,110

(22) PCT Filed: Dec. 19, 2011
(86) PCT No.: PCT/EP11/73300

§ 371 Date: Oct. 4, 2013

Related U.S. Application Data
(60) Provisional application No. 61/457,077, filed on Dec. 22, 2010.

(51) Int. Cl. A61K 31/55; A61K 31/357; A61K 31/496; A61K 31/506; A61K 31/538; A61K 31/4439; A61K 31/416; A61K 31/4725; A61K 31/4188; A61K 31/165; A61K 31/451

(52) U.S. Cl. CPC A61K 31/55; A61K 31/165; A61K 31/357; A61K 31/496; A61K 31/506; A61K 31/451; A61K 31/4439; A61K 31/416; A61K 31/4725; A61K 31/4188; A61K 31/538
USPC 514/213.01; 540/593; 564/161; 514/617; 549/362; 514/452; 544/360; 514/253.01; 544/319; 514/269; 546/206; 514/319; 544/327; 514/256; 546/279.1; 514/343; 548/362.1; 514/406; 546/15; 514/278; 544/284; 514/266.21; 548/301.1; 514/389; 544/105; 514/230.5; 435/7.21

(57) ABSTRACT

An in vitro method of screening candidate compounds for the preventive or curative treatment of rosacea is described. The method can include determining the capacity of a compound to modulate the expression or activity of the Transient Receptor Potential (TRPs), as well as the use of modulators of the expression or activity of this transcription factor for the treatment of rosacea. The method can also include in vitro diagnosis or prognosis of this pathology.

### BACKGROUND

Rosacea is a common, chronic and progressive inflammatory dermatosis related to vascular relaxation. It mainly affects the central part of the face and is characterized by redness of the face or hot flushes, facial erythema, papules, pustules, telangiectasia and sometimes an eye injury, also called ocular rosacea. In serious cases, particularly in men, the soft tissue of the nose may swell and produce a bulbous swelling known as rhinophyma. Rosacea generally occurs from the ages of 25 and 70, and it is much more common in individuals with a light complexion. It affects more particularly women, although this condition is generally more serious in men (Rosacea: Diagnosis and Management, Frank C. Powell, InformaHealthcare; New-York 2009.) Rosacea is chronic and persists for years with periods of exacerbation and remission. Rosacea was originally called ‘acne rosacea’ because its papules (points of slight raising of the skin) and its inflammatory pustules (pus scabs) greatly resemble those of common acne.

The result of this facial vascular anomaly is a permanent oedema of the dermis which may accompany an increased colonization with Demodex folliculorum, a parasite usually found in the follicles of the face.

Many factors may also be involved without necessarily inducing this condition. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

According to the National Rosacea Society, rosacea may be classified into four subtypes plus one variant (Erythematotelangiectatic rosacea, papulopustular, phymatous, eyepiece and a variant called granulomatous rosacea).

These subtypes are listed bellow.

Subtype 1: Eythematotelangiectatic Rosacea.

Subtype 1 is characterized by flushing and persistent central facial erythema. Telangiectases are common but not essential for the diagnosis. Central facial edema, burning sensations and scales are also reported symptoms. Conventionally, the patients have erythrosis spasms due to the sudden dilation of the arterioles of the face, which then take on a congestive, red appearance. These spasms can be triggered by emotions, meals and temperature changes.

Subtype 2: Papulopustular Rosacea.

Subtype 2 is characterized by persistent central facial erythema with transient papules or pustules or both in a central facial distribution. However, papules and pustules also may occur periorificially (that is, they may occur in the perioral, perinasal, or periocular areas). The papulopustular subtype resembles acne vulgaris, except that comedones are absent. Burning sensations may be reported by patients with papulopustular rosacea. This subtype has often been seen after or in combination with subtype 1, including the presence of telangiectases. The telangiectases may be obscured by persistent erythema, papules, or pustules. Some patients may also have red cheeks and forehead oedema.

Subtype 3: Phymatous Rosacea.

Subtype 3 includes skin thickening, irregular surface nodularities. Rhinophyma is the most common presentation, but phymatous rosacea may occur in other locations, including the chin, forehead, cheeks, and ears. Patients with this subtype may also have enlarged and prominent follicle openings. This subtype has frequently been observed after or in combination with subtypes 1 or 2, including persistent erythema, telangiectases, papules, and pustules. In the case of rhinophyma, these additional stigmata may be especially pronounced in the nasal area.

Subtype 4: Ocular Rosacea.

The diagnosis of ocular rosacea should be considered when a patient's eyes have one or more of the following signs and symptoms : watery or bloodshot appearance (interpalpebral conjunctival hyperemia), foreign body sensation, burning, dryness, itching, light sensitivity, blurred vision, telangiectasia of the conjunctiva and lidmargin, or lid and periocular erythema, blepharitis, conjunctivitis, Meibomian gland dysfunction. These signs or symptoms occur before, during or after the onset of cutaneous signs. Ocular rosacea is most frequently diagnosed when cutaneous signs and symptoms of rosacea are also present. However, skin signs and symptoms are not a prerequisite to the diagnosis, and limited studies suggest that ocular signs and symptoms may occur before cutaneous manifestations in up to 20% of patients with ocular rosacea.

Granulomatous Rosacea.

There is also a granulomatous variant of rosacea, characterized by yellow, brown or red, indurated papules or nodules, and monomorphic damage in papules. Other signs of rosacea may also occur.

Of course, the pathological manifestations of rosacea vary depending on the subtype of the disease. However patients may have characteristics of different subtypes at the same time. It is also known that the disease does not necessarily progress from a subtype to another (Wilkin et al., 2002, J. AM. Acad. Dermatol. Vol. 46, pages 584-587).

Several treatments exists, but there is always a need of novel compounds as well as new diagnostic methods and new monitoring methods in order to treat more efficiently patients suffering from rosacea.

TRPs

The family of closely related cation channels, denoted Transient Receptor Potential (TRP) are known to be molecular sensors for distinct pain, temperature, chemaesthesis, and taste modalities. The first implication of TRP channels in pain and sensation was emphasized by the identification of TRPV1 at the genetic and functional level (Caterina et al; 1997). TRPV1 (Transient Receptor potential cation channel, subfamily V, member 1) is a “ligand-gated non-selective cation channel” activated by various physical and chemical stimuli, exogenous and endogenous such as a temperature above 43° C., acidic pH, capsaicin (a component of red hot pepper and black pepper), ethanol and camphor. TRPV1 is therefore a polymodal nociceptive receptor and can be the sensor of various stimuli responsible of episodes of flush and erythema in rosacea (heat, food and hot or spicy drinks, alcoholic solutions). The activation of TRPV1 by the different stimuli, and therefore the Ca²⁺ entrance, induces the liberation of neuropeptides such as substance P, CGRP and PACAP, well know to induce neurogenic inflammation.

In human, 6 distinct receptors have been identified. Four of them, including TRPV1, TRPV2, TRPV3, TRPV4, have been identified to be expressed in sensory neurons and involved in nociception, thermosensation and chemaesthesis.

The table below is an overview of the expression of the TRP channels by sensory neurones or cells associated with sensory neurones that are involved in nociception, thermosensation and chemaesthesis. Thermo- and chemosensory properties referred to have been delineated by studying heterologously expressed TRP channels. The list of chemicals activating TRP channels is not complete.

| TRP Channel | Pain modality | Thermosensation | Chemaesthesis |
|---|---|---|---|
| TRPV1 | Heat | >42° C. | Capsaicin (red pepper), resinferatoxin (Euphorbia), |
| | Acidosis | | gingerol and zingerone (ginger), piperine (black pepper), |
| | Chemaesthetc | | eugenol (clove), camphor, vanillatoxins 1-3 (tarantula), |
| | (irritant) pain | | ethanol, acid (pH<6) |
| TRPV2 | Heat | >52° C. | Δ⁹ = tetrahydrocannabinol (Δ [symbol] = THC) |
| TRPV3 | Chemaesthetc | >33° C. (warmth) | Carvacrol (oregano), eugenol, thymol (thyme), vanillin |
| | (irritant) pain | | (vanilla), camphor, menthol (mint) |
| TRPV4 | Acidosis | >25-34° C. (warmth) | Acid (pH <6) |
| TRPM6 | Cold | <25° C. | Menthol, iclan, geraniol, L-carvone, isopulegol, linalool, |
| | Chemaesthetc | | |
| | (irritant) pain | | |
| TRPA1 | Chemaesthetc | <17° C. | Allicin and diallyl disulphide (garlic), |
| | (irritant) pain | | allyl isothiocyanate (mustard, horseradish, wasabi), |
| | | | cinnamaldehyde (cinnamon) carvacrol, |
| | | | gingerol, eugenol, icilin, acrotein, formaldehyde, |
| | | | methyl salyciate, Δ9 - THC |

Dhaka et al. (2006) and Bandel et al. (2007) describe the different subtypes of TRPs [symbol]
indicates data missing or illegible when filed

The invention relates to the use of TRP modulators and especially antagonists, diagnostic application and monitoring application involving the TRPs, in order to treat, diagnose and monitor rosacea in patients.

Also the invention relates to screening methods useful for determining the capability of a compound to inhibit TRP activity.

Diagnostic Applications:

A first aspect of the invention is an in vitro method of diagnosis or of monitoring the development of rosacea in a subject, comprising comparison of the expression at the mRNA or protein levels of the TRPs in a subject's biological sample, relative to a control subject.

The expression of the protein can be determined by an assay of the TRPs, classically but non limitative ways could be an immunohistochemical test or immunoassay, for example by ELISA assay.

In order to measure the expression of the gene or the amount of corresponding mRNA, a man skilled in the art could use any known method in this field.

Within the scope of diagnosis, the ‘control’ subject is a ‘healthy’ subject.

Within the scope of monitoring the development of rosacea, the ‘control subject’ refers to the same subject at a different time, which preferably corresponds to the start of the treatment (T0).

By measuring the difference in expression or activity of the TRPs, it is notably possible to monitor the efficacy of a treatment, in particular, a treatment with a modulator of the TRPs, as envisaged below, or by other treatments prescribed for rosacea patients.

Monitoring Applications:

Another aspect of the present invention features an in vitro method of determination of a subject's likelihood of developing rosacea, comprising comparison of the expression at the mRNA or protein levels of the TRPs in a subject's biological sample, relative to a control subject

Once again, the expression of the protein can be determined by an assay of the TRPs, classically but non limitative ways could be an immunohistochemical test or immunoassay, for example by ELISA assay.

In order to measure the expression of the gene, or the amount of corresponding mRNA, a man skilled in the art could use any known method in this field.

The subject tested is, in this case, an asymptomatic subject, not displaying any skin disorder associated with rosacea.

The ‘control’ subject, in this method, means a ‘healthy’ subject or reference population. Detection of this susceptibility makes it possible to start preventive treatment and/or increased monitoring for the signs associated with rosacea.

In these methods of in vitro diagnosis or prognosis, the biological sample tested can be any sample of biological fluid or a sample from a biopsy.

Preferably the sample can, however, be a preparation of skin cells, obtained for example by biopsy.

Modulators and Their Uses:

Another important aspect of the invention relates to modulators of TRPVs to target neurogenic inflammation and their use in the treatment or prevention of rosacea

These modulators of the TRPs may be used for the preparation of a medicinal product intended for the preventive and/or curative treatment of rosacea.

Thus, a method of preventive and/or curative treatment of rosacea is exemplified here, the said method comprising the administration of a therapeutically effective amount of a modulator of the TRPs to a patient needing the said treatment.

Preferably, said modulators are inhibitors (or antagonists) of the TRPs.

Preferentially said modulators are antagonists of TRPV1 as they may hold promise in limiting the deleterious effects of rosacea symptoms.

The modulator could be formulated within pharmaceutical compositions, together with a pharmaceutically acceptable excipient.

These compositions can be administered either by the enteral, parenteral, or topical route.

Preferably, the pharmaceutical composition is applied topically.

For oral administration, the pharmaceutical composition can be in the form of tablets, capsules, sugar-coated pills, syrups, suspensions, solutions, powders, granules, emulsions, suspensions of microspheres or nanospheres or lipid or polymer vesicles providing controlled release.

For parenteral administration, the pharmaceutical composition can be in the form of solutions or suspensions for infusion or for injection.

For topical application, the pharmaceutical composition is more particularly useful for the treatment of the skin, mucosae and scalp and can be in the form of unguents, creams, milks, ointments, powders, impregnated tampons, solutions, gels, sprays, lotions or suspensions. It can also be in the form of suspensions of microspheres or nanospheres or lipid or polymer vesicles or polymer patches or hydrogels providing controlled release.

This composition for topical application can be in anhydrous form, in aqueous form or in the form of an emulsion.

In a preferred variant, the pharmaceutical composition is in the form of a gel, a cream or a lotion.

As a non limitative example, the list in the table 1 below shows TRPV1 antagonist compounds that can be used for the treatment of rosacea, more particularly for the treatment of erythematotelangiectatic rosacea.

| | ACTIVITY OR |
|---|---|
| MOLECULES | REFERENCE |
| 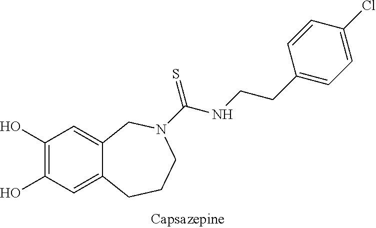 [SMILES: Oc1cc2c(cc1O)CN(C(=S)NCCc1ccc(Cl)cc1)CCC2] | EC₅₀ = 67 nM |
| 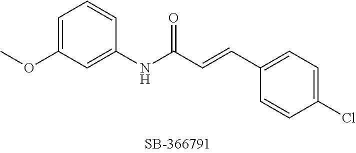 [SMILES: COc1cccc(NC(=O)/C=C/c2ccc(Cl)cc2)c1] | EC₅₀ = 39 nM |
| 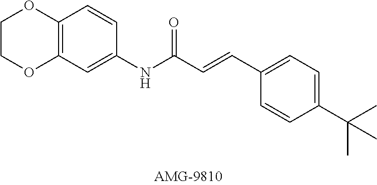 [SMILES: CC(C)(C)c1ccc(/C=C/C(=O)Nc2ccc3c(c2)OCCO3)cc1] | EC₅₀ = 1.9 nM |
| 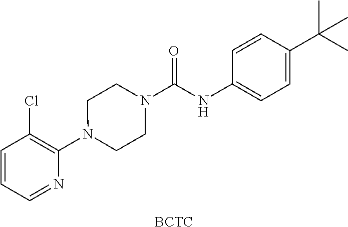 [SMILES: CC(C)(C)c1ccc(NC(=O)N2CCN(c3ncccc3Cl)CC2)cc1 ; "BCTC"] | EC₅₀ = 1.9 nM |
| 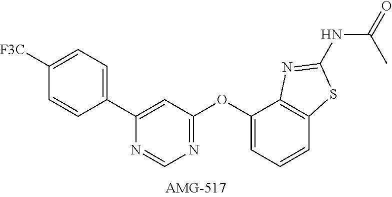 [SMILES: CC(=O)Nc1nc2c(Oc3cc(-c4ccc(C(F)(F)F)cc4)ncn3)cccc2s1] | EC₅₀ = 54 nM |
| 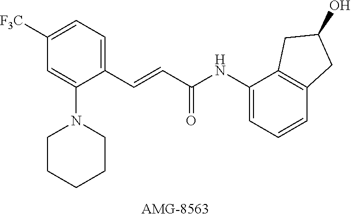 [SMILES: O=C(/C=C/c1ccc(C(F)(F)F)cc1N1CCCCC1)Nc1cccc2c1C[C@@H](O)C2] | IC₅₀ = 2.9 ± 0.49 nM (according to JPET 2008, 326, 218-229) |
| 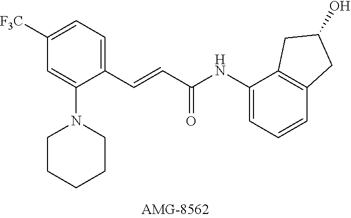 [SMILES: O=C(/C=C/c1ccc(C(F)(F)F)cc1N1CCCCC1)Nc1cccc2c1C[C@H](O)C2] | IC₅₀ = 1.75 ± 0.48 nM (according to JPET 2008, 326, 218-229) |
| 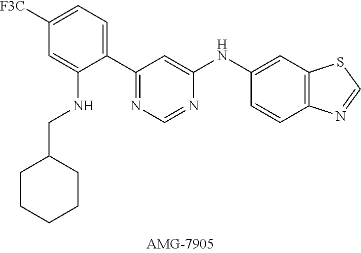 [SMILES: FC(F)(F)c1ccc(-c2cc(Nc3ccc4ncsc4c3)ncn2)c(NCC2CCCCC2)c1] | IC₅₀ = 39 ± 17 nM (according to JPET 2008, 326, 218-229) |
| 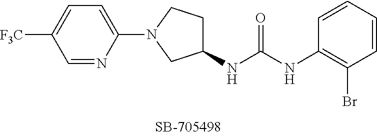 [SMILES: O=C(Nc1ccccc1Br)N[C@@H]1CCN(c2ccc(C(F)(F)F)cn2)C1] | pKi = 7.6 (according to JPET 2007, 321, 1183-1192) |
| 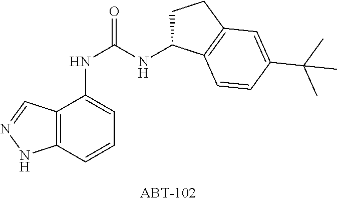 [SMILES: CC(C)(C)c1ccc2c(c1)CC[C@H]2NC(=O)Nc1cccc2[nH]ncc12] | IC₅₀ = 7 nM (according to Drug Discovery Today, January 2009, Volume 14, Numbers ½, 56-67) |
| 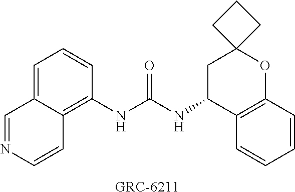 [SMILES: O=C(Nc1cccc2cnccc12)N[C@@H]1CC2(CCC2)Oc2ccccc21 ; "GRC-6211"] | WO 2007042906 |
| 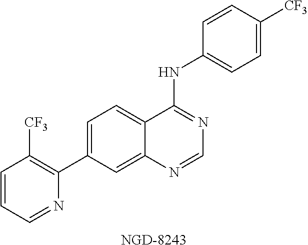 [SMILES: FC(F)(F)c1ccc(Nc2ncnc3cc(-c4ncccc4C(F)(F)F)ccc23)cc1 ; "NGD-8243"] | WO 2003062209 |
| 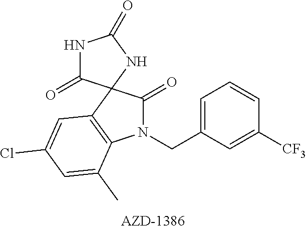 | WO 2007091948 |
| 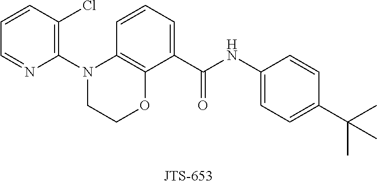 [SMILES: CC(C)(C)c1ccc(NC(=O)c2cccc3c2OCCN3c2ncccc2Cl)cc1] | WO 2006006741 |

In order to further illustrate the present invention and the advantages thereof, the following specific examples are given, it being understood that same is intended only as illustrative and in nowise limitative.

In said examples to follow, all parts and percentages are given by weight, unless otherwise indicated.

### EXAMPLE 1

### Table 1: TRPV1 Antagonists

Antagonist activity was tested on HEK293 cells over-expressing human TRPV1, in presence of capsaicin. Cells were incubated with the fluorescent calcium indicator dye, and intracellular calcium concentration ([Ca2+]i) was measured on specific instrument by detecting the fluorescence changes.

TRPV1 antagonist activity of compounds was further evaluated in a relevant model of neurogenic skin inflammation induced by a unique topical application of a TRPV1 agonist, resiniferatoxin RTX, on the back of SKH1 mice.

| | P (kinetic) |
|---|---|
| Study 1 | |
| RTX 0.03% versus RTX 0.03% + CZP 5% | *0.0444 |
| RTX 0.03% versus RTX 0.03% + AMG- | ***0.006 |
| 9810 5% | |
| Study 2 | |
| RTX 0.03% versus RTX 0.03% + CZP 5% | ***0.0002 |
| RTX 0.03% versus RTX 0.03% + AMG-517 | ***<0.0001 |
| 1% | |

### EXAMPLE 2

Table 2: TRPV1 antagonist activity of compounds is evaluated in a relevant model for neurogenic skin inflammation. Adult female hairless SKH1 mice aged 7-9 weeks are obtained from Charles River (France). Anaesthesia with ketamine/xylazine is performed 10 minutes before treatment and body temperature is maintained at 35° C.-37° C. using an automated heating pad. A skin area of 1.8×1.5 cm on the back of the mouse is divided in two regions of interest. One region is treated with the vehicle, the other one with RTX 0.03%. The compounds to be tested are applied 4 minutes before the single topical application of RTX on the two regions. Each mouse is its own control. The skin blood perfusion is evaluated every 2 minutes on the back of the mouse with a Laser Doppler perfusion imaging PIM3 (Perimed, France). Prior to treatment, scans are performed and measurements of blood perfusion change are made over 20 minutes. At the end of the study, mice are euthanized by cervical dislocation. Images are analysed and the yield of two parameters, the erythematous surface and the blood perfusion intensity ratio, is calculated as a relative area of vasodilatation (mm²). For each mouse, the value of the control area is removed from the value of the treated area. The statistical analysis performed is based on a repeated analysis of variance (P) of the relative area of vasodilatation. This analysis allowed the comparison of the group effect on the kinetic of the vasodilatation.

Results:

CZP (for capsazepine) and AMG-9810 used at the dose of 5% inhibits the vasodilatation induced by the topical application of RTX 0.03% on the back of SKH1 mice. AMG-517, at a dose of 1%, inhibits the vasodilatation induced by the topical application of RTX 0.03% on the back of SKH1 mice.

### EXAMPLE 3

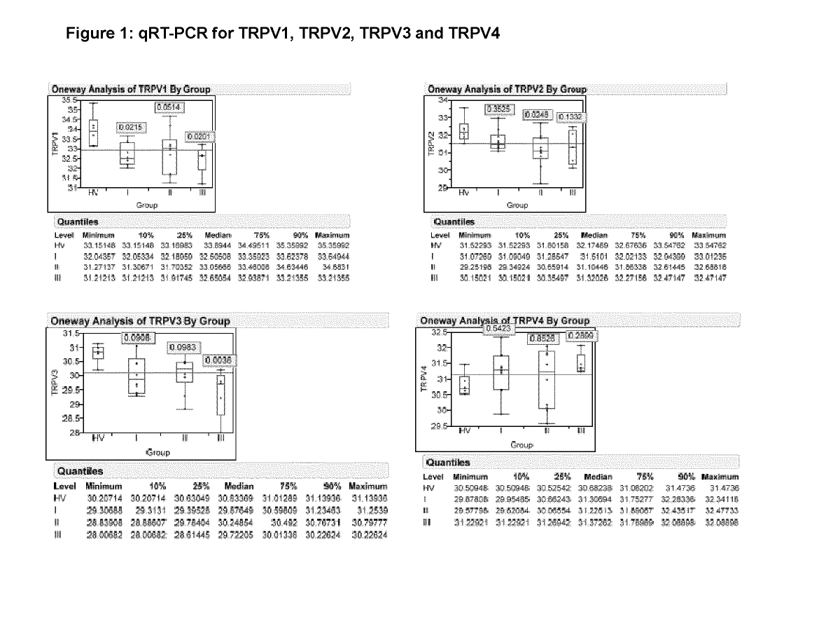

### Expression of TRPVs by qRT-PCR (FIG. 1)

Tissue collection:

Skin from healthy patients was obtained following plastic surgery (n=12; face). Skin biopsies of rosacea patients with rosacea subtype I (n=11), II (n=11) and III (n=6) were performed, in accordance with good clinical practice. The clinical diagnosis of rosacea subtypes was performed according to the classification of Wilkin et al., 2002, J. Am. Acad. Dermatol. Vol 46, pages 584-587. Permission was given by ethical committees in accordance with the ethical standards of the declaration of Helsinki.

mRNA expression of Transient Receptor Potential Cation Channels: TRPV1, TRPV2, TRPV3 and TRPV4 was evaluated using semi-quantitative PCR technology (qRTPCR-Taqman Low Density Arrays).

Total RNA was extracted using RNeasy extraction kits (Qiagen Inc.) according to manufacturer's protocol. RNA Quantity was measured using Quant-it RNA assay kit (Molecular Probes) and the quality was monitored by following the electrophoresis behaviour of RNA using a 2100 Bioanalyser (Agilent). 800 ng of extracted RNA of good quality [RNA indication number (RIN)≧7] was then used for synthesizing cDNA using high capacity cDNA archive kits (Applied Biosystems).

TaqMan Low Density Array (TLDA) Analysis

Gene expression analysis was performed using TLDA arrays containing PCR primers for genes of interest and housekeeping genes. A triplicate determination was performed for each sample.

The following ready-to-use TaqMan® Gene Expression Assays were used:

TRPV1: Hs00218912_m1

TRPV2: Hs00275032_m1

TRPV3: Hs00376854_m1

TRPV4: Hs00222101_m1

GAPDH: Hs99999905_m1

ACTB: Hs99999903_m1

HPRT: Hs99999909_m1

Synthesized cDNA (50 ng of cDNA per column) was added to the PCR master mix, and the mixture was loaded by centrifugation into the wells of the array containing the lyophilized primer sets (Applied Biosystems). The wells were sealed and the PCR reactions performed on ABI 7900HT (Applied Biosystems). PCR threshold cycle (Ct) numbers at which the fluorescent signal of the generated nascent DNA exceeds a threshold value were determined. The Ct number of a given gene in a given sample was normalized by first subtracting the average Ct of the housekeeping genes (GAPDH, ACTB, HPRT) in the same sample, and then adding back the average Ct of the housekeeping genes across all samples.

Statistical Analysis

The fold modulation of gene expression of rosacea samples versus samples of healthy volunteers was defined as 2 (mean CtHV-mean CtRo), with CtHV and CtRo depicting the Ct values of healthy volunteers and rosacea samples, respectively. To identify genes that were significantly modulated in the different Rosacea subtype samples, one-way ANOVA with Benjamini-Hochberg multiplicity correction was performed using JMP 7.0.1 (SAS Institute) and irMF 3.5 (National Institute of Statistical Sciences, NISS) software.

Legend FIG. 1:

Normalised Ct values of individual genes (TRPV1, TRPV2, TRPV3 and TRPV4) in healthy volunteers and in the three subtypes of rosacea patients are shown. The ends of the box indicate the lower and upper 25% quartiles. The line across the middle of the box identifies the median sample value. The width of the box is proportional to the number of observations. The central line in each subfigure identifies the global mean. P values of the comparisons of gene expression levels in the different subtypes of rosasea versus healthy volunteers are indicated.

Results:

The mRNA of TRPV1 is significantly up-regulated in the different subtypes of rosacea.

The mRNA of TRPV2 is significantly up-regulated in the subtypes II of rosacea, and a trend of up-regulation is also observed in subtypes I and III.

The mRNA of TRPV3 is significantly up-regulated in the subtype III of rosacea, and a trend of up-regulation is also observed in subtypes I and III.

The mRNA of TRPV4 is not modulated in rosacea but its expression is clearly demonstrated in the human skin with Ct reflecting a medium level of detection.

### EXAMPLE 3

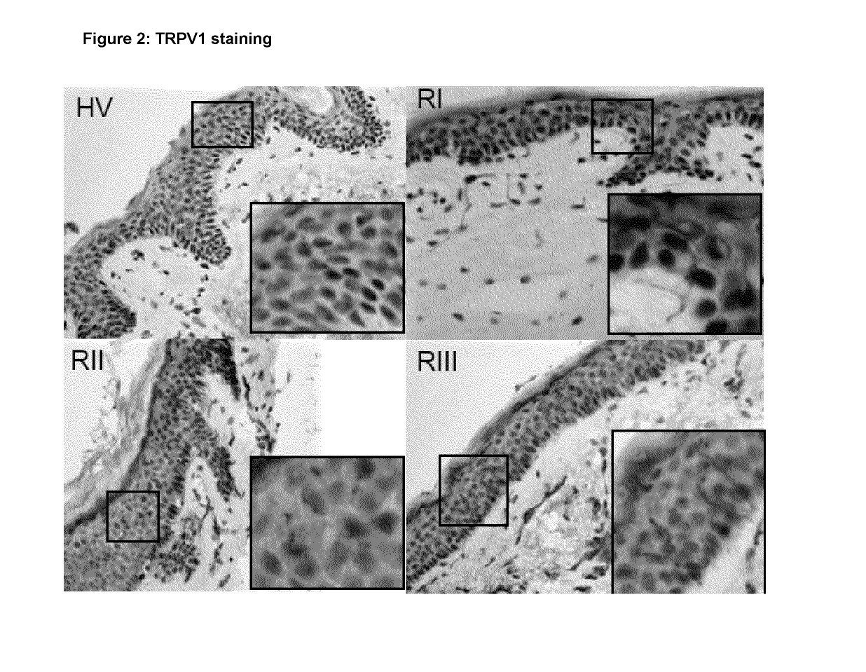

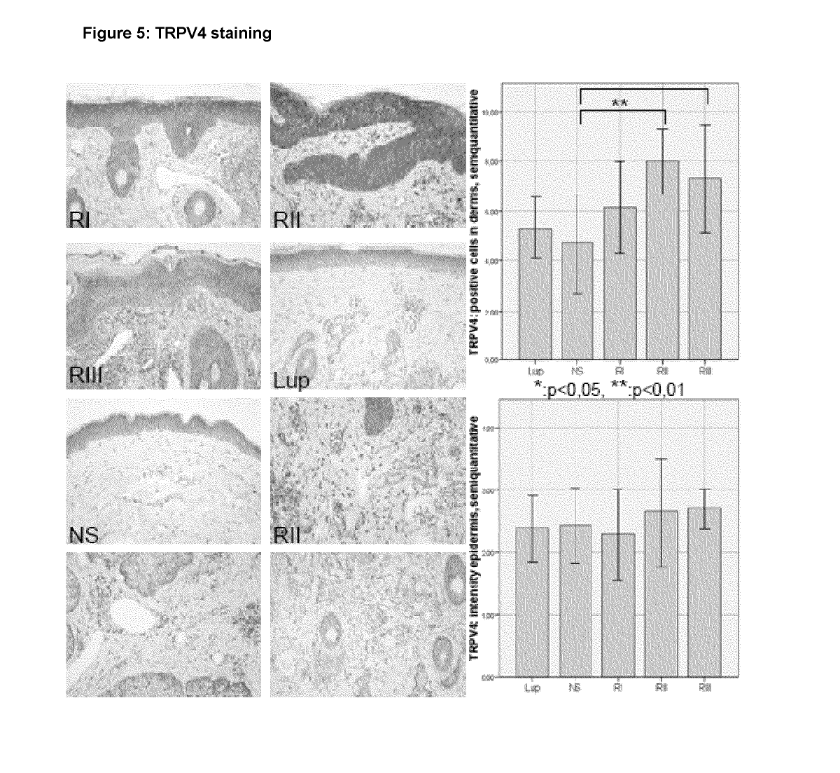

### In Situ Expression of TRPVs: Immunohistochemistry for TRPVs in Human Healthy and Rosacea Skin (FIGS. 2, 3, 4 and 5)

Tissue Preparation and Immunohistochemistry:

Skin from healthy patients was obtained from face-lift surgery (n=3). Skin biopsies from rosacea patients with rosacea subtype I (n=2), II (n=1) and III (n=1) were performed in accordance with good clinical practice.

Skin samples were immediately frozen in liquid nitrogen and cut after mounting in cryo-embedding compound with a cryostat. Frozen sections of 7 to 10 μm thickness were postfixed with cold acetone for 30 minutes then rehydrated in PBS buffer for 10 minutes before immunostaining.

TRPV1 staining was performed with Anti-Vanilloid Receptor-1 antibody (Sigma ref. V2764; 1/3000) using Discovery XT (Ventana Medical System, Roche) platform. ChromoMap red kit (Ventana Medical System, Roche) was used as antigen-antibody detection system using a secondary antibody UltraMap anti-rabbit AP (Ventana Medical System, Roche) conjugated with alkaline phosphatase. Hematoxylin II (Ventana Medical System, Roche) counterstaining was performed after the immunostaining. Slides were mountained using Cytoseal 60 mounting medium (Microm). All sections were immediately observed and photographed with a microscope (Axiolmager, Zeiss).

TRPV2, TRPV3, TRPV4 :

Result FIG. 2:

TRPV1 as shown in FIG. 2 is strongly expressed (red staining) in sensory nerve fibers both in the basal and the differentiated layers of the epidermis on skin samples. Qualitatively, we can observe an increase in the number of fibers expressing TRPV1 in the sample of rosacea type II (RII) and type III (RIII) compared to rosacea type I (RI) and healthy volunteers (HV).

TRPV2 :

As shown in FIG. 2

TRPV3

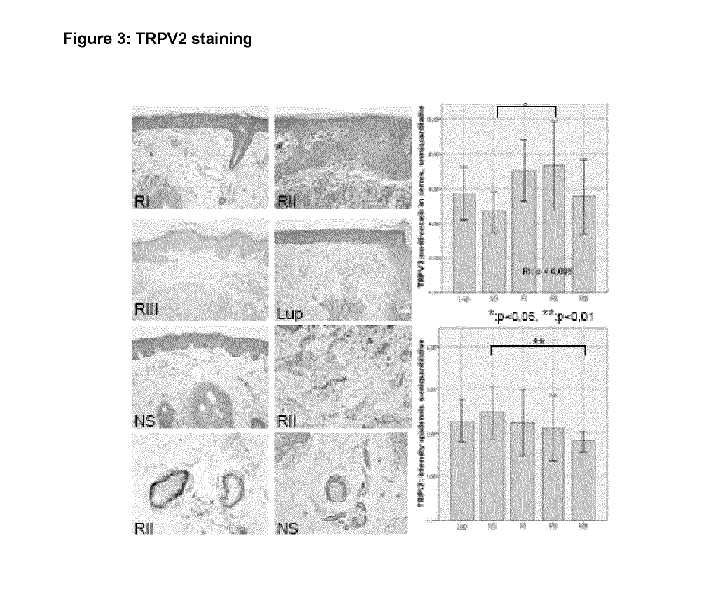

As shown in FIG. 3

Comment:

TRPV4:

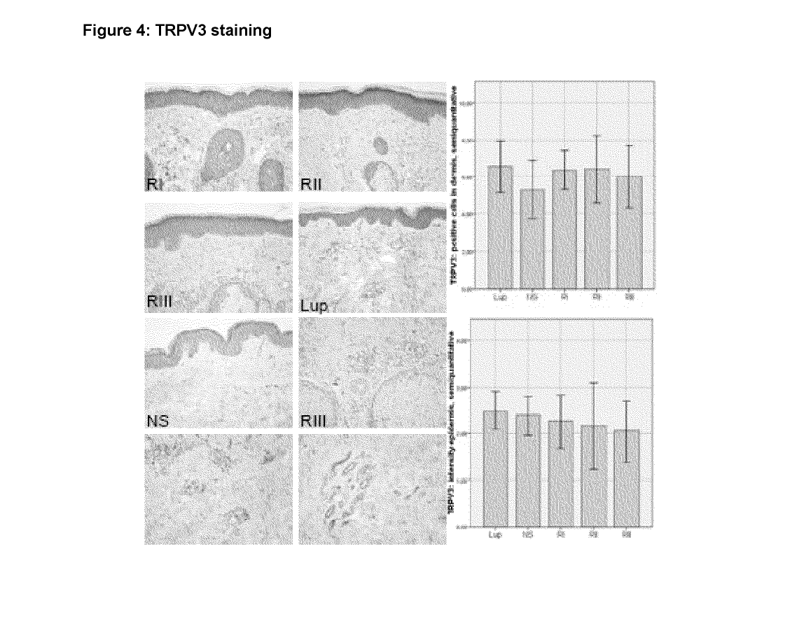

As shown in FIG. 4

Comment:

### EXAMPLE 4

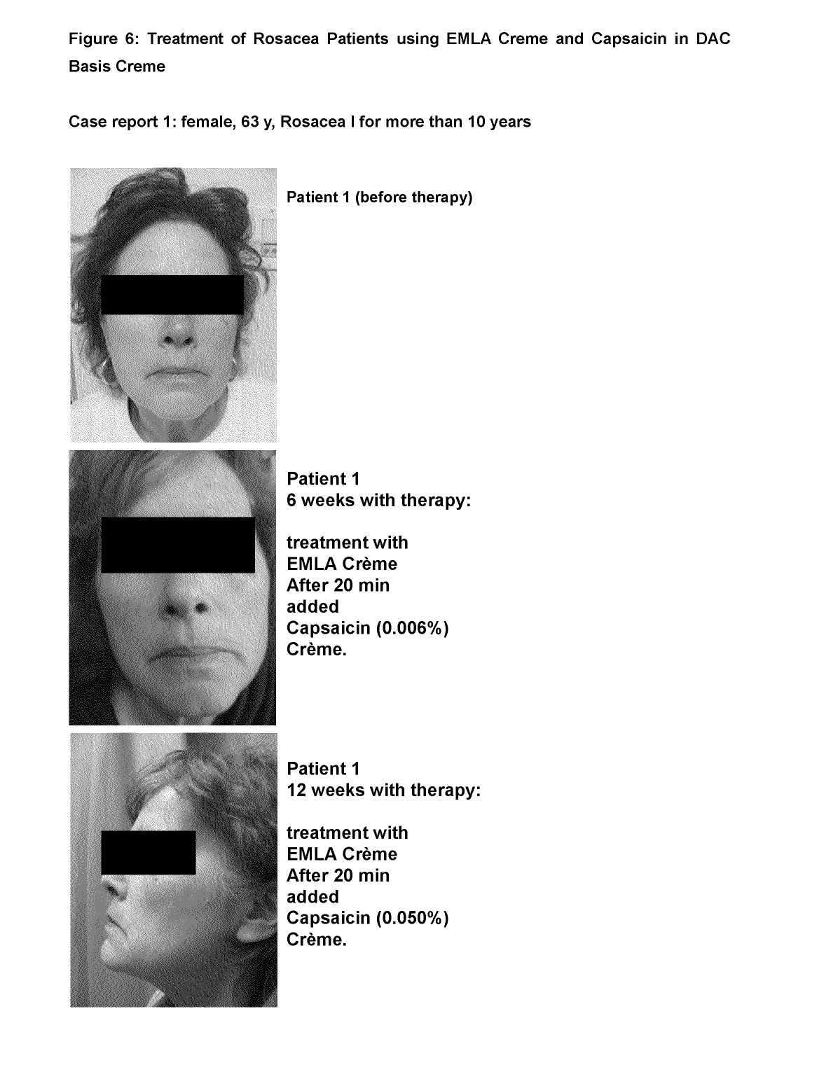

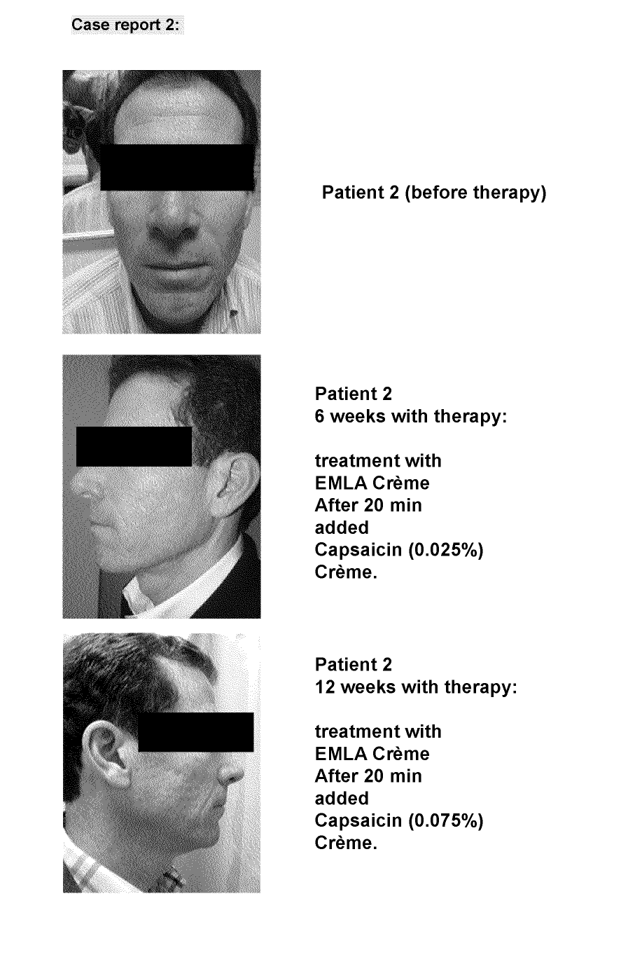

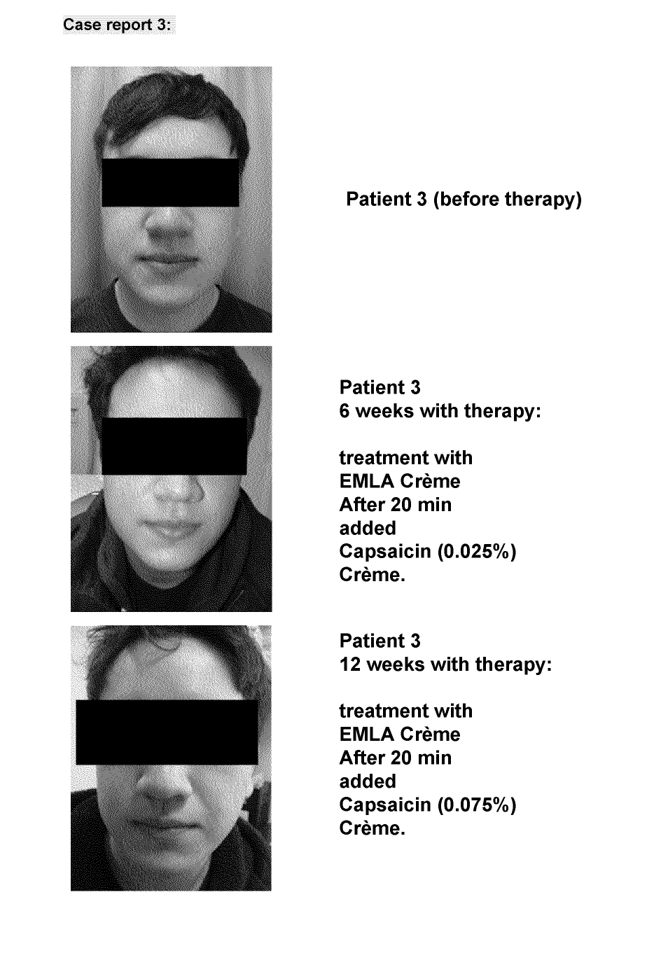

### Cases Reports—Modulator of TRPV1 as Efficient to Treat Rosacea Patient (FIG. 6)

Capsaicin, a TRPV1 agonist, is known to inhibit neurogenic inflammation after chronic application.

When Capsaicin is applied at the beginning, it induces stinging and burning. Such application may be beneficial for the long-term treatment of rosacea, but the combination with a compound may be beneficial. This would diminish or eliminate the discomfort which may be associated with the adverse effects of capsaicin treatment (burning, stinging, itching). Thus, it can be anticipated that nerval paralysis (anesthesia) induced by a local anesthetic (Na-channel blocker) may be beneficial to minimize adverse effects of topical capsaicin and maximize the effects of capsaicin to reduce the symptoms of rosacea, especially erythema. EMLA Creme (Lidocaine 2.5%/prilocaine 2.5%) is a combination of two effective and potent local anaesthetics which is regularly used for anesthesia before local skin surgery, wound debridement or before needle application in children.

In our rosacea patients, subtype I (erythema, edema, teleangiectasia), EMLA creme was applied to the erythematous skin for 30 min. After 30 min, capsaicin Creme (0.006-0.075%) was applied to the same sites of the erythematous rosacea. Capaicin was dissolved in DAC Basis Creme.

CONCLUSION of Case reports: Patients with subtype 1 of rosacea (erythemato-teleangiectatic) respond well to treatment with EMLA Creme (lidocaine 2.5%, prilocaine 2.5%) combined with Capsaicin Ointment (0.006-0.075% in DAC Basis Creme) when the skin is not too sensitive for topical application.

1. A medicament comprising a modulator of TRPs for treating rosacea.

2. The medicament as defined by claim 1, wherein the modulator comprises an antagonist of the TRPs.

3. The medicament as defined by claim 1, wherein the TRPs are selected from the group consisting of TRPV1, TRPV2, TRPV3 and TRPV4.

4. The medicament as defined by claim 1, wherein the TRP is TRPV1.

5. The medicament as defined by claim 1, wherein the TRP is TRPV2.

6. The medicament as defined by claim 1, wherein the TRP is TRPV3.

7. The medicament as defined by claim 1, wherein the TRP is TRPV4.

8. The medicament as defined by claim 1, wherein the rosacea is selected from the group consisting of erythematotelangiectatic rosacea, papulopustular rosacea, phymatous rosacea, ocular rosacea, and granulomatous rosacea.

9. The medicament as defined by claim 1, wherein the rosacea is rythematotelangiectatic rosacea.

10. An in vitro method of diagnosing or monitoring the development of rosacea in a subject, the method comprising comparing expression or activity of TRPs, or expression of its gene or activity of at least one of the promoters thereof, in a subject's biological sample with the expression or activity level in a biological sample of a control subject.

11. A regime or regimen for the treatment of rosacea, the regime or regimen comprising administering to a subject in need of such treatment, for such period of time as required to elicit the desired response, a thus effective amount of a modulator of TRPs.

12. The regime or regimen as defined by claim 11, wherein the TRPs are selected from the group consisting of TRPV1, TRPV2, TRPV3 and TRPV4.

13. The regime or regimen as defined by claim 11, wherein the TRP is TRPV1.

14. The regime or regimen as defined by claim 11, wherein the TRP is TRPV2

15. The regime or regimen as defined by claim 11, wherein the TRP is TRPV3.

16. The regime or regimen as defined by claim 11, wherein the TRP is TRPV4.

17. The regime or regimen as defined by claim 11, wherein the rosacea is selected from the group consisting of erythematotelangiectatic rosacea, papulopustular rosacea, phymatous rosacea, ocular rosacea, and granulomatous rosacea.

18. The regime or regimen as defined by claim 11, wherein the rosacea is erythematotelangiectatic rosacea.
